(19) United States
(12) Patent Application Publication
(10) Pub. No.: US 2007/0232984 A1
(43) Pub. Date: Oct. 4, 2007

(54) HAND-HELD ELECTRICAL STIMULATION DEVICE

(76) Inventors: Michael Lovell, Pittsburgh, PA (US); Marlin H. Mickle, Pittsburgh, PA (US)

Correspondence Address: REED SMITH LLP, P.O. BOX 488, PITTSBURGH, PA, 15230-0488, US

(21) Appl. No.: 11/693,641

(22) Filed: Mar. 29, 2007

Related U.S. Application Data
(60) Provisional application No. 60/788,571, filed on Mar. 30, 2006.

(51) Int. Cl. A61N 1/30

(52) U.S. Cl. 604/20

(57) ABSTRACT

Apparatuses and methods for the safe and efficient electrical stimulation of tissue. The apparatuses of the present invention are preferably hand-held and are capable of providing electrical waveforms that are effective in promoting wound healing, improving circulation, stimulating peripheral nerves, administering pharmaceutical compounds via electrophoresis, and electroporating DNA into tissue. The present invention also generally contemplates the safe and efficient transfection of DNA into mammalian tissue via electroporation. Particularly, apparatuses of the present invention are useful in the delivery of DNA vaccines and in gene therapy. The present invention also preferably provides for the direct measurement of in vivo tissue resistance. Tissue resistance measurements may then be used to adjust stimulation intensity during electroporation or electrostimulation, so as to avoid damaging tissue during operation of the unit.

### CROSS-REFERENCE TO RELATED APPLICATION

This application claims the benefit under 35 U.S.C. §119(e) of the earlier filing date of U.S. Provisional Application Ser. No. 60/778,571 filed on Mar. 30, 2006.

### GOVERNMENT RIGHTS

This invention was made with United States Government support in the form of Grant Nos. CA 74918, DK 54225, AR 45925, and DK 44935 from the National Institute of Health. The United States Government may have certain rights in the invention.

### BACKGROUND OF THE INVENTION

1. Field of the Invention

The present invention relates to methods and apparatuses for the efficient and safe transfection of DNA into mammalian tissue via electroporation and for the electrical stimulation of tissue so as to promote improved circulation, wound healing, or peripheral nerve stimulation.

2. Description of the Background

Electrical stimulation of biological tissue has been an acceptable mode of medical therapy for many years. It is widely used in biomedical research as well as in diagnostic and clinical applications. Specifically, electrical stimulation has been used to promote nerve regeneration, wound healing, and for electrophoretic application of pharmaceutical compounds. In addition, improvement in circulation has also been reported as a result of electrical stimulation. Peripheral stimulation has further been shown to be effective in the treatment of chronic pain management. Stronger electric stimulation has also been used to transfer deoxyribonucleic acid (DNA) into cells through electroporesis as described more fully hereinbelow.

Following tissue damage, electrical signals are generated which are believed to initiate the wound healing process. It is thought that these electrical signals are instrumental in ensuring that the necessary cells are drawn to the wound location to bring about wound healing. Localized exposure to low levels of electrical current may mimic or augment these naturally-occurring electrical signals and has been shown to enhance the healing of soft tissue wounds in both human subjects and animals. In particular, external electrical stimulation has been reported to accelerate ulcer healing and result in a more robustly-healed wound. Dayton and Palladino in “Electrical Stimulation of Cutaneous Ulcerations—A Literature Review,” Journal of the American Podiatric Medical Association, 79(7):318, 1989, which is hereby incorporated by reference.

U.S. Pat. Nos. 5,433,735 and 4,982,742 (both of which are hereby incorporated by reference) disclose various electrical stimulation apparatuses and techniques for facilitating the regeneration and repair of damaged tissue. Additional physiological electrical stimulators are well known in the art with multiple commercial and experimental apparatuses being available. Many of those stimulators require wall-based electrical outlets to operate, thus limiting their utility in unconventional circumstances such as operation in remote or technologically-underdeveloped locations.

Molecular biological research has established that many diseases have a genetic component. As the genetic bases of diseases are elucidated, the potential benefit of gene therapy continues to grow. Typically, genetic-based treatment of diseases involves the introduction of exogenous DNA to cells in vivo. Two examples of gene therapy applications include gene replacement therapy and DNA-based vaccination.

Traditional live vaccines are not suitable for mass immunization efforts due to their toxicity, instability, and relatively high cost. One advantage of DNA-based vaccines is that expression of an antigen from exogenous DNA appears to result in the activation of all pathways of immunity, especially cytotoxic T-cell responses, which have been difficult to induce with standard protein vaccines. For viruses, including those which have caused problems for blood transfusion, DNA vaccination could easily be used for prevention of infection. In addition, plasmid DNA used in a DNA vaccine can be easily purified on a large scale by column chromatography and other well known procedures. Highly-purified DNA is free from hazardous contaminants and its quality is easily managed through well-developed molecular biological techniques. Further, DNA is much more stable than conventional vaccines and can be kept at room temperature for long periods of time.

One of the long-standing obstacles to the implementation of DNA vaccines is the safe and efficient delivery of exogenous DNA to cells in vivo. Gene delivery methods in vivo can be classified into viral and non-viral categories. Viral vectors are highly efficient in gene transfer. However, concerns remain regarding the safety of viral vectors in a clinical setting. Among non-viral techniques, intramuscular injection of DNA has been demonstrated to be a safe, simple, and inexpensive approach for gene delivery. However, this method also has several limitations. First, the level of gene expression is, in most cases, too low for efficient treatment of diseases or induction of appropriate immune responses. Second, there is high inter-individual variability in the level of the transferred gene expression. The reason for the lack of reproducibility is unknown, but it nevertheless presents an obstacle to clinical application of this technique. In addition, while early work into this technique was performed with rodents, primate muscle appears to be less efficient in its ability to take up injected DNA.

Transfection efficiency by electroporation is many fold greater than that of naked DNA injection, with minimal inter-individual variability. In addition, electrotransfer can achieve long-lasting expression in different tissue types of various species, including primates. Gene transfer by electroporation in vivo has been effective for introducing DNA into rat hepatocellular carcinomas, hepatocytes, mouse testes, melanoma, and skeletal muscle.

Electroporation employs large voltage fields to enhance the cellular uptake of extracellular molecules. The electrical stimulus causes membrane destabilization and subsequent formation of nanopores in the cellular membrane. In this permeabilized state, the membrane allows entry of various macromolecules, including DNA, to the cytoplasm. Electrotransfer efficiency depends strongly on the strength of the electric field. At low field strength, the plasma membrane of the cell is not sufficiently altered to allow passage of DNA. Typically, maximal levels of transient gene expression are reached using an electrical field of 250-750 V/cm. However, tissue damage has been observed at field strengths of >100 V/cm. In addition, higher field strengths (approximately 1500 V/cm) cause extensive and irreversible damage to tissue. The damage induced by electroporation represents a major obstacle for clinical gene therapy or gene-based vaccination applications.

In traditional clinical electroporation devices, a pair of calipers is typically used to pinch a patch of skin. The two calipers are then used to deliver the current that is used in generating the voltage field. A syringe needle or pair of syringe needles is typically inserted into the patch of skin and DNA is injected coincidentally with the electrical field. Because the calipers make high resistance contact with the skin, a great deal of current must be passed to generate a sufficient voltage field for electroporation to occur. Accordingly, tissue damage is encountered using this technique.

In other currently-employed electroporation devices, arrays of needles are inserted into tissue. Voltage fields are established among the various needles of the array. However, those approaches employ open circuitry in delivering the voltage to the tissue, i.e., there is no assessment of the resistance of the tissue in which the voltage field is being applied. Accordingly, voltage levels are set to very high levels to ensure efficient electroporation (approximately 1500 V) and are not changed during the course of the protocol. At these voltage levels, significant tissue damage may occur. In addition, many devices of the prior art employ specialized needles and, therefore, cannot use readily available, off-the-shelf components.

An additional complication that confronts electroporation approaches is the variable resistance of tissue. During the course of electroporation, the resistance of the tissue that is being stimulated changes. Accordingly, the current that is required to maintain the same voltage field will also change. Without repeated assessment of the resistance of tissue and appropriate compensation of the stimulation, the efficiency of electroporation may decrease during the stimulation protocol.

Typically, electroporation and electrical stimulation devices require substantial desktop hardware for the operation of the system. Such devices also typically require electricity from an electrical outlet. Together, these properties limit the flexibility in implementing an electroporation device. Further, it is very difficult, if not impossible, to take traditional electroporation or electrical stimulation devices out into the field for applications such as immunization. Given the target population for immunization is often in rural areas without electrical power, such limitations can be significant.

Thus, there has been a long-standing need in the medical field for a device that provides for electrical stimulation of tissue in a flexible and safe manner. The medical field also lacks a device that facilitates the safe and efficient electroporation of DNA into tissue. Preferably, such a device would be capable of assessing the resistance of the tissue into which the DNA is to be injected, thus allowing an electric feedback-based approach to electroporation. Such a device would also preferably be hand-held and capable of being battery-operated, so as to be ergonomic and portable.

### BRIEF DESCRIPTION OF THE DRAWINGS

For the present invention to be clearly understood and readily practiced, the present invention will be described in conjunction with the following figures, wherein like reference characters designate the same or similar elements, which figures are incorporated into and constitute a part of the specification, wherein:

FIG. 1A depicts a side view of an apparatus of the present invention;

FIG. 1B depicts a rear view of an apparatus of the present invention;

FIG. 2 displays internal components of an apparatus of the present invention;

FIG. 3 is a schematic of circuitry that may be used within the context of the present invention; and

FIG. 4 shows the layout of a syringe within an electrode cartridge of the present invention.

### SUMMARY OF THE INVENTION

The present invention preferably includes apparatuses and methods that provide for the safe and efficient electrical stimulation of tissue. Apparatuses of the present invention used to accomplish that task are preferably hand-held and portable. In some embodiments, apparatuses of the present invention are adapted to provide electrical waveforms that are effective in promoting wound healing, improving circulation, stimulating peripheral nerves, administering pharmaceutical compounds via electrophoresis, and electroporating DNA into tissue.

The present invention, in accordance with at least one presently-preferred embodiment, generally contemplates the safe and efficient transfection of DNA into mammalian tissue via electroporation. Particularly, some presently-preferred embodiments of the present invention are useful in the delivery of DNA vaccines and in gene therapy.

### DETAILED DESCRIPTION OF PREFERRED EMBODIMENTS OF THE PRESENT INVENTION

It is to be understood that the figures and descriptions of the present invention have been simplified to illustrate elements that are relevant for a clear understanding of the invention, while eliminating, for purposes of clarity, other elements that may be well known. The detailed description will be provided hereinbelow with reference to the attached drawings.

The present invention preferably includes apparatuses and methods that provide for the safe and efficient electrical stimulation of tissue. Apparatuses of the present invention are preferably hand-held and portable. The apparatuses of the present invention are particularly useful for improving wound healing and/or circulation, stimulating peripheral nerves, administering pharmaceutical agents through electrophoresis, and electroporating DNA into tissue.

While specific embodiments of the present invention will be discussed hereinbelow, one of skill in the art will recognize multiple ways of implementing the inventive concepts displayed by the present invention. The specific examples that are described are, therefore, intended to be illustrative and not limiting.

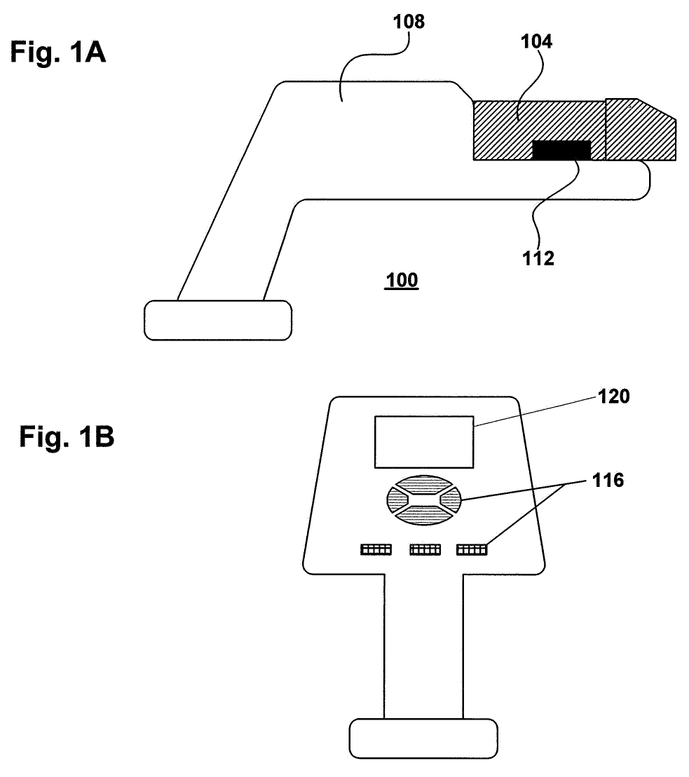

The apparatuses of the present invention are preferably hand-held and ergonomic. A presently-preferred embodiment of the present invention 100 is shaped similarly to a handgun and is shown in FIG. 1A. The embodiment rests comfortably in the hand of the user so that it could be used many times over the course of a day. Due, in part, to the reduced stimulation intensities that are required by the present invention, the present invention may be battery powered, thus making the apparatus portable.

In the presently-preferred apparatus of the present invention 100 shown in FIG. 1A the external portion of the apparatus preferably has two main components—a disposable electrode cartridge 104 and a hand-held stimulation system 108. The disposable electrode cartridge 104 is preferably adapted so as to snap quickly into and out of the housing of the stimulation system 108 using a snap lock system 112. The electrode cartridge 104 may include various designs of electrodes such as standard EEG-type electrodes or those disclosed in U.S. Pat. No. 6,907,294 which is incorporated herein by reference. Alternatively, the apparatuses of the present invention may be adapted to accept electrode arrays. The specific electrode type will easily be selected by one of skill in the art and will depend on the specific application for which the invention is being employed. For example, if the present invention is being used for wound healing, then the electrode could be designed to cover at least a significant portion of the wound to be treated.

In a presently-preferred embodiment, the electrodes are fully contained within the electrode cartridge and remain covered until the system is engaged. The electrode cartridge will preferably be prepackaged and sterilized. For health and safety considerations, the electrode cartridge should only be used for a single patient.

Presently-preferred apparatuses of the invention also include a cartridge insertion lever which is shown as a snap lock 112 on FIG. 1A. The cartridge insertion lever 112 is adapted to reside at least partially within the chamber that accepts the electrode cartridge 104. The snap lock 112 also preferably does not allow the apparatus 100 to be activated unless an electrode cartridge 104 is loaded. In that sense, the cartridge insertion lever 112 acts as a safety switch for the system 100. In addition, the cartridge insertion lever 112 may be used to eject a used electrode cartridge 104 at the completion of a stimulation paradigm.

The embodiment of the present invention 100 shown in FIG. 1A is preferably activated through user interface buttons as shown 116, for example, in FIG. 1B. A screen-based, menu-driven interface may be employed within the context of the present invention. Preferably, an LCD 120 displays various parameters of the stimulation protocol. The user of the present apparatus employs push buttons 116 to select and change those parameters. The screen 120 may also be used to display the resistance of the tissue, the status of the protocol, the amount of DNA injected, and/or other attributes of the patient being treated.

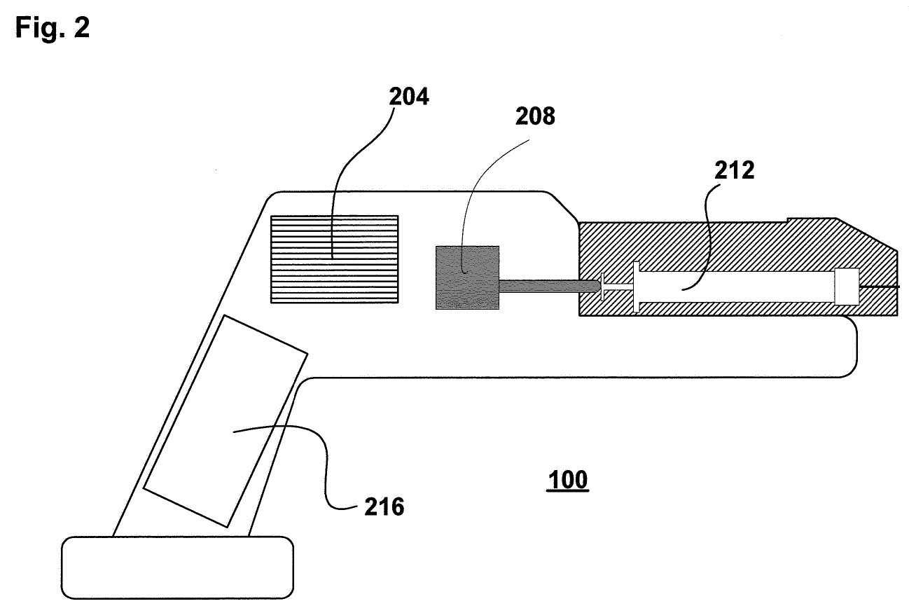

A schematic view of the major internal components of an embodiment of the present invention 100 used for electroporation is shown in FIG. 2. In presently-preferred embodiments of the present invention, the system is powered by a 7.2-volt battery 204, though other appropriate self-contained power sources may also be employed. In electroporation applications, the battery 204 may be used to power a linear actuator 208 that is responsible for injecting solution containing DNA into the region of tissue from the syringe 212. The battery 204 is also capable of powering the user interface and the circuitry, shown as a printed circuit board 216, used to generate the stimulation pattern. While the battery 204 is shown in the upper body of the housing in FIG. 2, the battery 204 may also be in other locations, such as in the base of the apparatus.

The apparatuses of the present invention further may also include a printed circuit board (FIG. 2). The printed circuit board is primarily responsible for generating the electrical signals required to drive the linear actuator (if used), to generate the appropriate electrical field at the electrodes, and to present the user with information about the status of the stimulation protocol via the user interface. One of skill in the art would recognize multiple ways of implementing such circuitry. The circuit board preferably contains the circuitry required to generate the electrical field at the electrodes so as to achieve a desired stimulation pattern.

In the particular embodiment shown in FIG. 2, the apparatus is adapted for use in electroporation. In that context, the apparatus includes an electrode cartridge that preferably contains syringes. In addition, a linear actuator is preferably present to allow for injection of solution into the tissue. In embodiments where simple electrical stimulation is to be employed, the linear actuator may optionally be present.

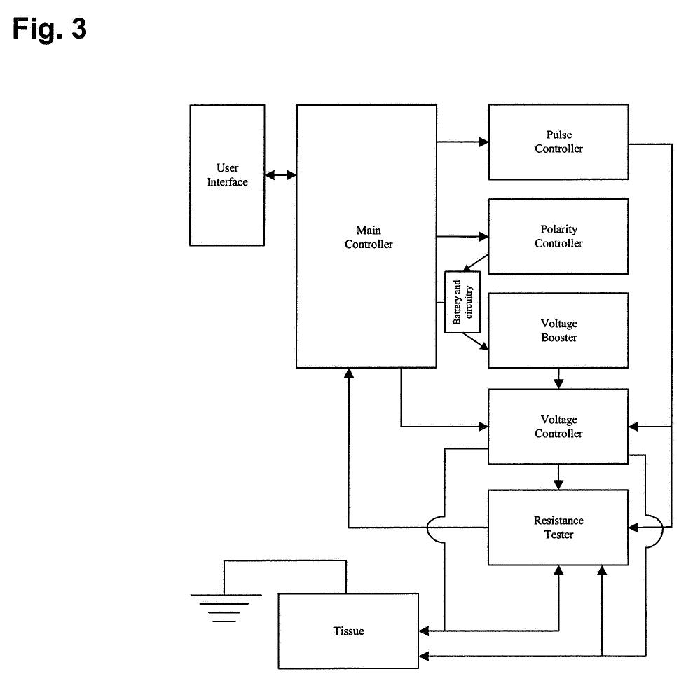

A schematic of the circuitry that may be used to implement the present invention is shown in FIG. 3. The user interface is employed to interface with the main controller, which is located on the printed circuit board (FIG. 2). The main controller sends data regarding the stimulation pattern to the voltage controller. The voltage of the signal may then be boosted by a voltage booster to the appropriate value. The specific voltage pattern may be formed by integrated circuitry present on the circuit board according to the parameters set by the user. The polarity and pulse sequence is conditioned by the polarity and pulse controllers, respectively, and the signal is delivered to the muscle tissue via an electrode. During this time, the electrode may also be employed to test the resistance of the tissue. That information may be fed back into the main controller to adjust the next voltage pulse. Preferably, the resistance measuring portion of the circuit includes an assembly of known resistors connected in series and in parallel with the muscle tissue via the electrode, as described below. The apparatus may also be adapted for the delivery of current-based stimulation patterns. One of skill in the art would easily recognize how to implement such a system within the context of the present invention.

As shown in FIG. 2 and discussed above, the apparatuses of the present invention may be adapted for use in electroporation. In such applications, the apparatus is adapted to accept syringes that also act as electrodes. In a presently-preferred embodiment of the present invention, a single one-milliliter syringe is included in the electrode cartridge. The syringe preferably includes DNA solution for injection into the region of tissue. In such embodiments, the electrode cartridge may be referred to as a syringe cartridge. When syringes are used as electrodes as in the case of electroporation, the cartridge also may contain a metal ring or other metallic conductor at the outer edge to establish electrical contact with the needles of the syringes if employed. Alternatively, electrical contact may be established between the circuitry of the present invention and syringe needles by conductive tape, conductive gel, conductive foam, a spring-loaded clamp, or a similar mechanism.

In electroporation applications, the stimulation is preferably implemented in a closed loop feedback design. In other words, the present apparatus is capable of measuring the resistance of the tissue, and thus assessing the quality of the electrode implantation. The circuit then capable of adjusting the current or voltage appropriately so that an appropriate voltage level may be maintained in the tissue.

One advantage provided by the present invention is the measurement of tissue resistance. The syringe needles that penetrate into the region of tissue are capable of not only delivering the electrical stimulation required for electroporation, but also measuring the resistance of the tissue. By measuring the resistance of tissue, more appropriate stimulation may be delivered during electroporation events. In addition, patient-specific data may be collected for later use in cases where multiple electrical stimulation sessions are required.

By measuring the resistance of the tissue, the present invention provides for the dramatic improvement in the safety of electroporation. As stated above, traditional devices set the voltage at which electroporation occurs to very high levels to ensure that electroporation occurs efficiently. However, such strong stimulation may damage the tissue. In contrast, the present invention is capable of adjusting the stimulation intensity in response to the measured resistance of the tissue. Accordingly, much lower stimulation intensities may be employed to achieve the same efficiency of transfection compared to the prior art while, at the same time, minimizing tissue damage.

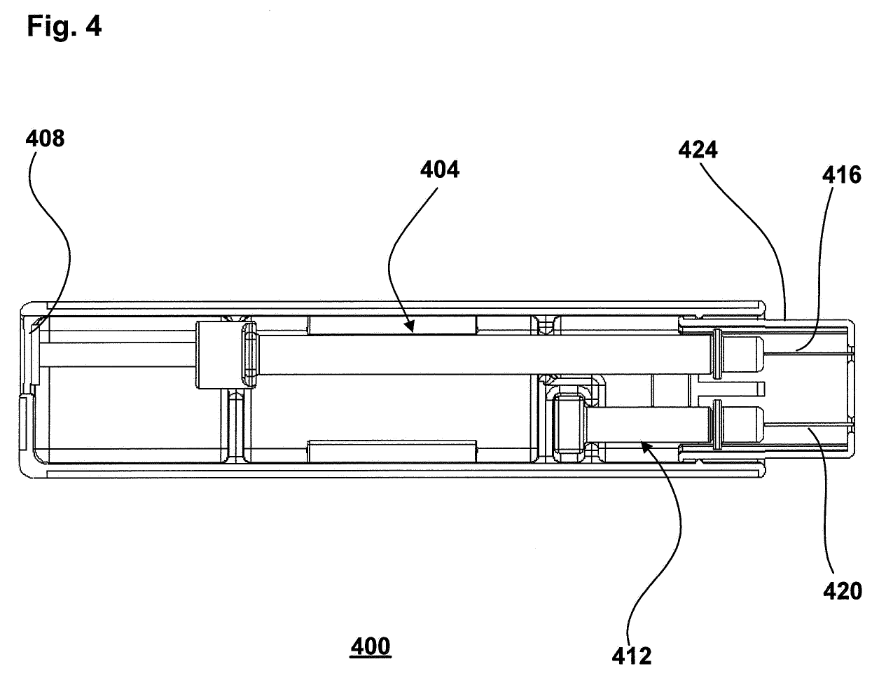

In embodiments where electroporation is to be performed, such as FIG. 2, the linear actuator may be used to inject DNA into the target tissue (FIG. 4). Once activated, the linear actuator extends to depress the plunger on the syringe that contains the DNA solution, thus injecting the solution into the tissue. After the syringe is fully injected and the electrical fields have been applied, the linear actuator retracts so that it can be employed for the next injection. The particular signals that are to be sent to activate the linear actuator will, of course, depend on the particular linear actuator that is employed.

The internal structure of a presently-preferred embodiment of a syringe cartridge 400 is displayed in FIG. 4. The syringe cartridge 400 includes a syringe 404 that is capable of holding solution that contains DNA. In the displayed position, the syringe 404 is full and the plunger 408 is not depressed. The syringe cartridge 400 may also include a second syringe 412 that is not used to deliver solution to the patient. Both syringes 404, 412 preferably include needles 416, 420 that penetrate the skin of the patient. The needles 416, 420 are preferably shielded from the external world by a retractable housing 424. In some embodiments, the retractable housing 424 is freely retracting. In other preferred embodiments, the depth of the housing 424 retraction may be set such that the needles 416, 420 of the syringes 404, 412 penetrate the tissue to a predetermined depth.

When the syringe cartridge 400 is attached to the injection system of the present invention and the retractable housing 424 is pressed against the skin of a patient, the housing 424 is depressed and the syringe needles 416, 420 extend from the housing 424 into the tissue of the patient. At that point in the injection process, the linear actuator depresses the plunger 408 injecting the DNA-containing solution into the patient. Following injection and electroporation, the injection apparatus is removed from the patient and the retractable housing 424 preferably extends to cover the syringe needles 416, 420.

The apparatuses of the present invention may be utilized in multiple contexts within the medical industry. For example, the apparatuses of the present invention could be used to deliver DNA vaccines to patients safely and effectively. Indeed, since DNA vaccines are much more stable than either protein or live-virus vaccines, the apparatuses of the present invention are ideally suited for vaccination against various types of biological challenges. In addition, the apparatuses of the present invention may be used for cancer treatment by utilizing syringes that can directly penetrate a tumor. In such a manner, anti-cancer genes may be introduced directly into a tumor. The apparatuses of the present invention are preferably light-weight and capable of being hand-held so as to promote their ease of use in the circumstance of mass sterilization. In other embodiments, the apparatuses of the present invention may be used to promote wound healing, peripheral nerve stimulation, or to promote circulation.

### EXAMPLE

The overall operation of a preferred apparatus according to the present invention proceeds as follows. When the apparatus is not loaded with a syringe cartridge, an appropriate message is displayed on the LCD screen. When a syringe cartridge is inserted into the apparatus, a circuit is initiated within the circuit board to allow the stimulation protocol to commence. The user may then select parameters for the stimulation waveform.

The user then takes the device and presses it into the deltoid muscle of a patient. The user assesses the resistance of the injection site by selecting that functionality via the menu on the rear of the apparatus. After confirming the successful impaling of the patient, the user then initiates the injection process by pressing the appropriate user interface button. An electrical signal is then sent to the linear actuator via the main controller. The linear actuator extends, depressing the syringe plunger and injecting the DNA-containing solution into the tissue. Once the injection protocol is initiated, a message is sent to the user on the LCD screen to indicate that the DNA injection process is occurring.

When the syringe plunger is fully depressed, the linear actuator has reached the appropriate extension, thus indicating that the DNA injection is complete. Alternatively, the plunger may be depressed by a specific amount to deliver a particular volume of fluid into the tissue. A series of voltage pulses is then delivered to the injection site through the syringe electrodes. For example, a train of six pulses of 50 V/cm may be delivered to the injection site at a frequency of 1 Hz.

Once the electrical stimulation cycle is complete, the linear actuator to its initial position. As the device is withdrawn from the patient's arm, the syringe needles are covered by the retractable housing within the syringe cartridge and a message is displayed to the user that the injection sequence is complete. The syringe cartridge may then be removed by unlocking the snap lock that had held it in place.

Those of skill in the art will recognize that numerous modifications of the above-described methods and apparatuses can be performed without departing from the present invention. For example, one of skill in the art will recognize that the apparatuses of the present invention may be implemented using various designs for the housing and a variety of implementations for the circuitry that may be employed within the context of the present invention.

1. An apparatus for the electrical stimulation of tissue, comprising:
an electrode cartridge;
a circuit adapted to produce an electrical stimulation protocol;
a battery; and
a housing, wherein said housing is adapted to accommodate said electrode cartridge, said circuit, and said battery.

2. The apparatus of claim 1, wherein said electrical stimulation protocol further comprises measurement of a resistance of said tissue.

3. The apparatus of claim 2, wherein said electrical stimulation protocol is a voltage protocol.

4. The apparatus of claim 2, wherein said housing is retractable.

5. The apparatus of claim 1, wherein said apparatus is hand-held.

6. The apparatus of claim 1, further comprising a user interface.

7. The apparatus of claim 6, wherein said user interface is adapted to allow a user to set an electrical stimulation pattern.

8. The apparatus of claim 2, wherein said electrical stimulation protocol is specifically adapted to improve wound healing, improve circulation, stimulate a peripheral nerve, administer pharmacological agents via electrophoresis, or administer DNA to said tissue via electroporation.

9. The apparatus of claim 1, wherein said electrode cartridge includes a syringe.

10. The apparatus of claim 9, further comprising a linear actuator adapted to depress said syringe.

11. The apparatus of claim 9, wherein said syringe includes a plunger, a chamber, and a first needle.

12. The apparatus of claim 11, wherein said electrode cartridge further comprises a second needle.

13. The apparatus of claim 12, wherein said first needle and second needle are adapted to assess resistance of said tissue.

14. The apparatus of claim 1, wherein said circuit comprises:
a main controller;
a pulse controller;
a polarity controller;
a voltage booster;
a voltage controller; and
a resistance tester.

15. The apparatus of claim 14, wherein said resistance tester includes a first electrode and a second electrode.

16. A method of delivering DNA to cells in a tissue using a portable electrical stimulation device, comprising the steps of:
measuring a resistance of said tissue;
administering a voltage protocol to said tissue, wherein voltage protocol is based on said resistance; and
injecting DNA into said tissue

17. The method of claim 16, wherein said measuring, administering, and injecting steps are performed by a single apparatus.

18. The method of claim 16, wherein a syringe is used to accomplish said injecting step.

19. The method of claim 18, wherein said syringe is used to measure said resistance.

20. The method of claim 18, wherein said syringe is used administer said voltage protocol.
